(19) United States
(12) Patent Application Publication
(10) Pub. No.: US 2024/0270651 A1
(43) Pub. Date: Aug. 15, 2024

(54) HEXAGONAL BORON NITRIDE POWDER AND METHOD FOR PRODUCING SAME, COSMETIC PREPARATION AND METHOD FOR PRODUCING SAME, AND QUALITY EVALUATION METHOD

(71) Applicant: DENKA COMPANY LIMITED, Tokyo (JP)

(72) Inventor: Ryuki MATSUI, Tokyo (JP)

(73) Assignee: DENKA COMPANY LIMITED, Tokyo (JP)

(21) Appl. No.: 18/568,152

(22) PCT Filed: Jun. 16, 2021
(86) PCT No.: PCT/JP2021/022903

§ 371 Date: Dec. 7, 2023

(51) Int. Cl. C04B 35/583; C04B 35/622; C04B 35/626

(52) U.S. Cl. CPC C04B 35/583; C04B 35/62222; C04B 35/6262; C04B 35/62655; C04B 35/62675; C04B 2235/386; C04B 2235/5409; C04B 2235/658

(57) ABSTRACT

A hexagonal boron nitride powder, in which a ratio of an oxygen content to a specific surface area (N) determined through nitrogen adsorption is 0.1 [g/100 m²] or less. A hexagonal boron nitride powder, in which a coating area proportion of an adhesive surface for which a ball number is 7 in an inclined ball tack test, specified in JIS Z 0237:2009, with an inclined plate with an inclination angle of 30 degrees is 77% or more when the hexagonal boron nitride powder is applied to and spread on the adhesive surface from one end to the other end of the adhesive surface using a coating plate at a rate of 1 cm/sec.

### TECHNICAL FIELD

The present disclosure relates to a hexagonal boron nitride powder and a method for producing the same, a cosmetic preparation and a method for producing the same, and a quality evaluation method.

### BACKGROUND ART

Boron nitride has lubricity, high thermal conductivity, insulation properties, and the like, and is used in a wide range of applications, including solid lubricants, mold release agents, fillers for resins and rubber, raw materials for a cosmetic preparation (also called a cosmetic), and insulating sintered bodies with heat resistance.

Examples of functions of the hexagonal boron nitride powder mixed into the cosmetic preparation include improving slipperiness, spreadability, and concealability of the cosmetic preparation, and imparting glossiness to the cosmetic preparation. In particular, hexagonal boron nitride powder has superior slipperiness compared to a talc powder and a mica powder, which have similar functions, and is therefore widely used in the cosmetic preparation that requires excellent slipperiness. Patent Literature 1 proposes that the ratio of shear stress to a pressing force be within a predetermined numerical range to improve slipperiness.

### CITATION LIST

### Patent Literature

[Patent Literature 1] Japanese Unexamined Patent Publication No. 2019-43792

### SUMMARY OF INVENTION

### Technical Problem

To meet the increasingly high level of customer demands for a cosmetic preparation, there has been a demand for further improvement in the properties of raw materials used in the cosmetic preparation. Therefore, for example, it is thought that it is necessary for raw materials used for foundation and the like to have superior spreadability. On the other hand, hexagonal boron nitride powder is likely to form agglomerated lumps, which is thought to affect spreadability. Therefore, the present disclosure provides a hexagonal boron nitride powder that can produce a cosmetic preparation with excellent spreadability, and a method for producing the same. In addition, the present disclosure provides a cosmetic preparation with spreadability using the above-described hexagonal boron nitride powder, and a method for producing the same. In addition, the present disclosure provides a quality evaluation method that can simply evaluate the quality of a hexagonal boron nitride powder or a powdery composition containing the same.

### Solution to Problem

According to an aspect of the present disclosure, there is provided a hexagonal boron nitride powder, in which a ratio of an oxygen content to a specific surface area (N) determined through nitrogen adsorption is 0.1 [g/100 m²] or less. Such a hexagonal boron nitride powder has a small oxygen content per unit surface area. For this reason, for example, moisture is less likely to be adsorbed onto the surface of the hexagonal boron nitride particles in an air atmosphere. In addition, static electricity generated on the surface can be reduced. It is inferred that, due to these factors, aggregation of the hexagonal boron nitride powder would be suppressed, resulting in excellent spreadability. Such a hexagonal boron nitride powder is suitable for use as a raw material for a cosmetic preparation.

According to another aspect of the present disclosure, there is provided a hexagonal boron nitride powder, in which a coating area proportion of an adhesive surface for which a ball number is 7 in an inclined ball tack test, specified in JIS Z 0237:2009, with an inclined plate with an inclination angle of 30 degrees is 77% or more when the hexagonal boron nitride powder is applied to and spread on the adhesive surface from one end to the other end of the adhesive surface using a coating plate at a rate of 1 cm/sec. This is provided that the above-described coating area proportion is a ratio of a coating area to a total area of the adhesive surface in a central area when 0.1 g of the hexagonal boron nitride powder is applied to and spread on the adhesive surface. The central area is a 10 mm square area 10 mm away from one end of the adhesive surface. The entire hexagonal boron nitride powder is arranged on one end side of the adhesive surface between virtual extension lines of a pair of sides that partition the central area along the direction in which the hexagonal boron nitride powder is applied and spread.

Since the above-described hexagonal boron nitride powder has a large coating area proportion of the adhesive surface, it has excellent spreadability and fineness. Further, the adhesive surface for which a ball number is 7 in the above-described inclined ball tack test is similar to human skin in terms of spreadability and conformability of the hexagonal boron nitride powder. For this reason, the hexagonal boron nitride powder having a large coating area proportion on the above-described adhesive surface has excellent spreadability and fineness when particularly applied to human skin. Such a hexagonal boron nitride powder is suitable for use as a raw material for a cosmetic preparation. In the hexagonal boron nitride powder, a ratio of an oxygen content to a specific surface area (N) determined through nitrogen adsorption may be 0.1 [g/100 m²] or less.

The above-described hexagonal boron nitride powder may have a specific surface area (H) of 0.8 [m²/g] or less which is determined through water vapor adsorption. Since moisture in atmospheric air is less likely to be adsorbed onto such a hexagonal boron nitride powder, aggregation of the hexagonal boron nitride powder is further suppressed and the hexagonal boron nitride powder has superior spreadability.

A ratio of the specific surface area (H) determined through water vapor adsorption to the specific surface area (N) determined through nitrogen adsorption may be 0.2 or less. Since such a hexagonal boron nitride powder can sufficiently suppress adsorption of moisture, aggregation of the hexagonal boron nitride powder is further suppressed and the hexagonal boron nitride powder has superior spreadability. The oxygen content of the above-described hexagonal boron nitride powder may be 0.15 mass % or less. Thus, adsorption of water vapor can be further suppressed, and spreadability can be further improved.

The above-described hexagonal boron nitride powder may be used as a raw material for a cosmetic preparation. Since the above-described hexagonal boron nitride powder has excellent spreadability, it is suitable for use as a raw material for a cosmetic preparation.

A cosmetic preparation according to one aspect of the present disclosure contains any of the hexagonal boron nitride powder described above. Aggregation of the above-described hexagonal boron nitride powder is suppressed, and the hexagonal boron nitride powder has excellent spreadability. For this reason, the cosmetic preparation containing such hexagonal boron nitride powder has excellent spreadability.

Since the above-described hexagonal boron nitride powder has excellent spreadability and fineness when applied to human skin, the cosmetic preparation containing the hexagonal boron nitride powder also has excellent spreadability and fineness when applied to human skin.

A method for producing a hexagonal boron nitride powder according to an aspect of the present disclosure includes: a firing step of firing a mixed powder containing hexagonal boron nitride and an aid at 1600° C. or higher and lower than 1900° C. in an atmosphere of an inert gas, ammonia gas, or a mixed gas thereof to obtain a fired product containing hexagonal boron nitride having higher crystallinity than the hexagonal boron nitride of the mixed powder; a purification step of pulverizing, washing, and drying the fired product to obtain a dry powder; and an annealing step of annealing the dry powder at 1900° C. or higher in an atmosphere of an inert gas, ammonia gas, or a mixed gas thereof.

In the above-described production method, the fired product containing hexagonal boron nitride having high crystallinity can be obtained by performing firing at the temperature of 1600° C. or higher and lower than 1900° C. using the aid. By pulverizing this fired product and then washing it, a residual aid and the like can be reduced and grain growth during subsequent annealing can be suppressed. After drying, the fired product containing hexagonal boron nitride which has already been crystallized is annealed at a temperature of 1900° C. or higher, which reduces the oxygen content by dispersing oxygen and oxygen-containing functional groups attached to the surface of primary particles while inhibiting grain growth of the particles. Since such a hexagonal boron nitride powder has a low oxygen content per unit surface area, moisture is less likely to be adsorbed onto the particle surface. In addition, static electricity generated on the surface can be reduced. It is inferred that, due to these factors, aggregation of the hexagonal boron nitride powder would be suppressed, resulting in excellent spreadability and fineness. Such a hexagonal boron nitride powder is suitable for use as a raw material for a cosmetic preparation.

The above-described production method may further include, before the firing step: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing low crystalline hexagonal boron nitride. The mixed powder in the firing step may contain the calcined product and the aid. In this manner, by performing calcination at a temperature lower than that in the firing step, a hexagonal boron nitride powder having a large specific surface area can be obtained. In addition, by performing calcination at a temperature lower than that in the firing step, a hexagonal boron nitride powder having superior spreadability and fineness can be obtained.

In the hexagonal boron nitride powder obtained through the above-described annealing step, a ratio of an oxygen content to a specific surface area (N) determined through nitrogen adsorption may be 0.1 [g/100 m²] or less.

In the hexagonal boron nitride powder obtained in the above-described production method, a coating area proportion of an adhesive surface for which a ball number is 7 in an inclined ball tack test, specified in JIS Z 0237:2009, with an inclined plate with an inclination angle of 30 degrees may be 77% or more when the hexagonal boron nitride powder is applied to and spread on the adhesive surface from one end to the other end of the adhesive surface using a coating plate at a rate of 1 cm/sec. The coating area proportion is a ratio of a coating area to a total area of the adhesive surface in a central area when 0.1 g of the hexagonal boron nitride powder is applied to and spread on the adhesive surface. The central area is a 10 mm square area 10 mm away from one end of the adhesive surface. The entire hexagonal boron nitride powder is arranged on one end side of the adhesive surface between virtual extension lines of a pair of sides that partition the central area along the direction in which the hexagonal boron nitride powder is applied and spread.

The adhesive surface for which a ball number is 7 in the above-described inclined ball tack test is similar to human skin in terms of spreadability and conformability of the hexagonal boron nitride powder. For this reason, the hexagonal boron nitride powder having a large coating area proportion on the above-described adhesive surface has excellent spreadability and fineness when particularly applied to human skin. Such a hexagonal boron nitride powder is suitable for use as a raw material for a cosmetic preparation.

A method for producing a cosmetic preparation according to an aspect of the present disclosure produces the cosmetic preparation using the hexagonal boron nitride powder obtained through any of the above-described production methods as a raw material. Aggregation of the hexagonal boron nitride powder obtained through the above-described production method is suppressed, and the hexagonal boron nitride powder has excellent spreadability. For this reason, the cosmetic preparation produced using such hexagonal boron nitride powder as a raw material has excellent spreadability.

A quality evaluation method according to an aspect of the present disclosure includes: a step of applying and spreading a hexagonal boron nitride powder or a powdery composition containing the same onto an adhesive surface using a coating plate; and a step of performing quality evaluation based on a coating area of the hexagonal boron nitride powder or the powdery composition containing the same on the adhesive surface. According to this quality evaluation method, it is possible to simply evaluate the quality such as spreadability and fineness of the hexagonal boron nitride powder.

The above-described adhesive surface may be composed of one side of carbon tape. Since the carbon tape is black in color, it is possible to determine the coating area proportion of the adhesive surface using the white hexagonal boron nitride powder with high accuracy. Accordingly, the quality of the hexagonal boron nitride powder can be evaluated with high accuracy.

The above-described adhesive surface may be an adhesive surface for which ball numbers are 6 to 8 in an inclined ball tack test, specified in JIS Z 0237:2009, with an inclined plate with an inclination angle of 30 degrees. Such an adhesive surface is similar to human skin in terms of spreadability and conformability of the hexagonal boron nitride powder. Accordingly, the method is useful as a method for evaluating the quality such as spreadability and fineness of cosmetic preparations and their raw materials that are applied to human skin.

### Advantageous Effects of Invention

In one aspect of the present disclosure, it is possible to provide a hexagonal boron nitride powder that can produce a cosmetic preparation with excellent spreadability, and a method for producing the same. In addition, it is possible to provide a cosmetic preparation with excellent spreadability using the above-described hexagonal boron nitride powder, and a method for producing the same.

In another aspect of the present disclosure, it is possible to provide a hexagonal boron nitride powder with excellent spreadability and fineness, and a method for producing the same. In addition, it is possible to provide a cosmetic preparation with excellent spreadability and fineness. In addition, it is possible to provide a quality evaluation method that can simply evaluate the quality of a hexagonal boron nitride powder.

### BRIEF DESCRIPTION OF DRAWINGS

FIG. 1 is a plan view of a quality evaluation device used in one example of a quality evaluation method.

FIG. 2 is a side view of the quality evaluation device of FIG. 1.

FIG. 3 is a view showing a measurement device for performing an inclined ball tack test.

FIG. 4 is a photograph showing a state after hexagonal boron nitride powders of Example 1 and Comparative Example 1 are applied to and spread on each adhesive surface.

### DESCRIPTION OF EMBODIMENTS

Hereinafter, embodiments of the present disclosure will be described. However, the following embodiments are merely examples for describing the present disclosure and are not intended to limit the present disclosure to the following contents.

In a hexagonal boron nitride powder of the present embodiment, a ratio of an oxygen content to a specific surface area (N) determined through nitrogen adsorption is 0.1 [g/100 m²] or less. The ratio may be less than 0.08 [g/100 m²]. The ratio may be less than 0.05 [g/100 m²] or may be less than 0.03 [g/100 m²]. By reducing the ratio, spreadability can be improved. The ratio may be, for example, 0.001 [g/100 m²] or more, or 0.005 [g/100 m²] or more. This can improve dispersibility in a polar solvent. For this reason, for example, in a case where the hexagonal boron nitride powder is used as a raw material for a cosmetic preparation, it is possible to produce the cosmetic preparation smoothly. An example of a range of the ratio may be 0.001 to 0.1 [g/100 m²].

The specific surface area (N) determined through nitrogen adsorption is a value measured using a commercially available specific surface area measurement device using nitrogen as an adsorption gas. The specific surface area (N) may be 0.5 [m²/g] or more, or may be 1 [m²/g] or more. By having a large specific surface area (N), primary particles can be made sufficiently small. This can improve adhesion to the skin and wrinkles. The specific surface area (N) may be 8 [m²/g] or less, or may be 6 [m²/g] or less. This makes it possible to sufficiently increase not only spreadability but also slipperiness. An example of a range of the specific surface area (N) may be 0.5 to 8 [m²/g].

The oxygen content may be 0.15 mass % or less, or may be 0.12 mass % or less. By lowering the oxygen content, adsorption of moisture onto the particle surface can be suppressed. In addition, static electricity generated on the particle surface can be reduced. Due to these factors, aggregation of the hexagonal boron nitride powder can be suppressed. The oxygen content may be 0.005 mass % or more, or may be 0.01 mass % or more. This can improve dispersibility in a polar solvent. For this reason, for example, in a case where the hexagonal boron nitride powder is used as a raw material for a cosmetic preparation, it is possible to produce the cosmetic preparation smoothly. The oxygen content can be adjusted by changing the firing temperature and time in a firing step and the annealing temperature and time in an annealing step. An example of a range of the oxygen content may be 0.005 to 0.15 mass %.

A specific surface area (H) determined through water vapor adsorption is a value measured using a commercially available specific surface area measurement device using water as an adsorption gas. That is, as this value increases, the amount of moisture adsorbed onto the particle surface increases. The specific surface area (H) may be 0.8 [m²/g] or less, or may be 0.6 [m²/g] or less. By having a low specific surface area (H), the adsorption of moisture onto the particle surface is suppressed, and aggregation of the hexagonal boron nitride powder is suppressed. The specific surface area (H) may be 0.1 [m²/g] or more, or may be 0.2 [m²/g] or more. This can improve dispersibility in an aqueous solvent. An example of a range of the specific surface area (H) may be 0.1 to 0.8 [m²/g].

A ratio of the specific surface area (H) determined through water vapor adsorption to the specific surface area (N) determined through nitrogen adsorption may be 0.2 or less or 0.17 or less. By reducing the ratio, the adsorption of moisture can be further suppressed. The lower limit of the ratio may be 0.01 or 0.03. This can improve dispersibility in a polar solvent. An example of a range of the ratio of the specific surface area (H) to the specific surface area (N) may be 0.01 to 0.2. By changing the time of the annealing step, the ratio of the specific surface area (H) to the specific surface area (N) can be adjusted. For example, by prolonging the time of the annealing step, the ratio of the specific surface area (H) to the specific surface area (N) can be reduced.

The hexagonal boron nitride powder according to the present embodiment is less likely to form an agglomerated lump and has excellent spreadability, and therefore, it is suitable for use as a raw material for a cosmetic preparation. That is, the present disclosure can also provide a method of using a hexagonal boron nitride powder as a raw material for a cosmetic preparation. A cosmetic preparation having excellent spreadability can cover a wider area of the skin when applied to and spread on the skin. Such a hexagonal boron nitride powder may have a coating area proportion of 80% or more or 90% or more as determined through a quality evaluation method below. An example of a range of the coating area proportion may be 80% to 99%.

A quality evaluation method according to one embodiment includes: a first step of applying and spreading a hexagonal boron nitride powder or a powdery composition containing the same onto an adhesive surface using a coating plate; and a second step of determining a coating area proportion of the hexagonal boron nitride powder on the adhesive surface.

The adhesive surface may be composed of one side of tape. For example, if the adhesive surface is composed of one side of carbon tape, the adhesive surface will be black in color, and therefore, it is possible to determine the coating area proportion of the white hexagonal boron nitride powder or powdery composition with high accuracy.

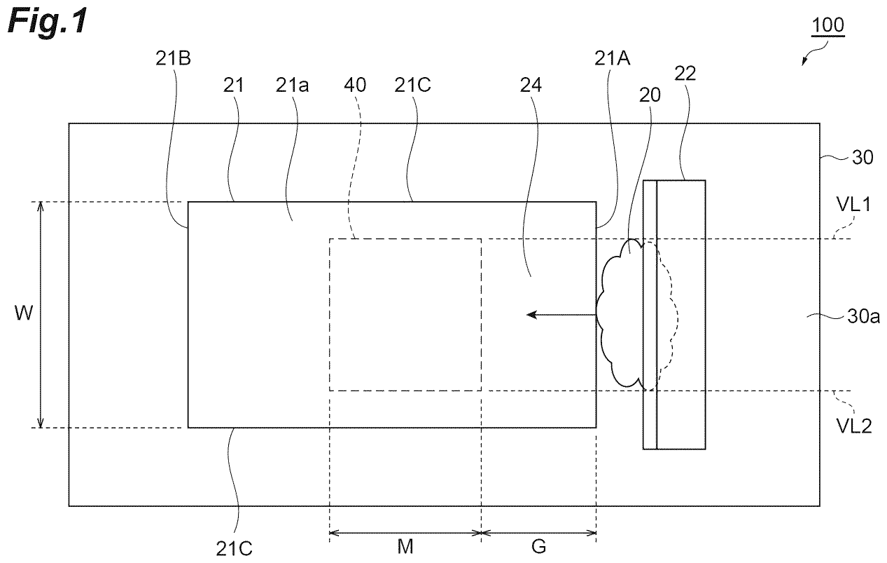

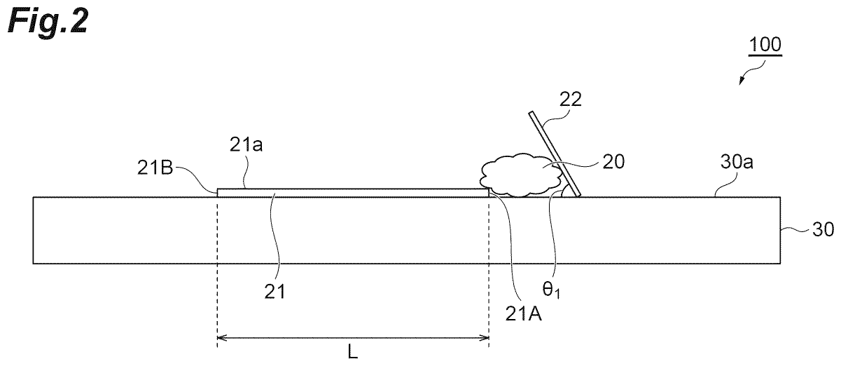

FIG. 1 is a plan view of a quality evaluation device used in one example of the above-described quality evaluation method. FIG. 2 is a side view of the quality evaluation device of FIG. 1. A quality evaluation device 100 includes: a pedestal 30; a tape 21 having an adhesive surface 21a pasted on an upper surface 30a of the pedestal 30; a powdery sample 20 placed on one end 21A side of the tape 21 (adhesive surface 21a); and a coating plate 22 of applying and spreading the sample 20 onto the adhesive surface 21a from one end 21A side to the other end 21B side.

The pedestal 30 can be used without particular limitation as long as it has the flat upper surface 30a. The coating plate 22 may have rigidity to such a degree that it does not deform when the sample 20 is applied and spread. The pedestal 30 and the coating plate 22 may be made of a resin or metal. The sample 20 may be a hexagonal boron nitride powder, or may be a powdery composition (for example, a cosmetic preparation) containing a hexagonal boron nitride powder.

The adhesive surface 21a of the tape 21 has a central area 40 in the center portion. The central area 40 is located in the center portion of the tape 21 (adhesive surface 21a) in the direction in which a pair of side portions 21C of the tape 21 (adhesive surface 21a) face each other. The central area 40 may have a square shape which is divided into four sides and of which each side has a length M. There may be a space 24 between the central area 40 and one end 21A of the adhesive surface 21a. If the coating area proportion in such a central area 40 is calculated, the variation in measurement values of the coating area proportion will be reduced, and the quality of the sample 20 can be evaluated with high accuracy. According to these quality evaluation device 100 and quality evaluation method, it is possible to evaluate the quality such as spreadability and fineness of the sample 20.

In the first step, the sample 20 is placed on the one end 21A side of the adhesive surface 21a. At this time, a part of the sample 20 may be prevented from adhering to the adhesive surface 21a. Subsequently, the coating plate 22 is moved at a predetermined speed in the arrow direction in FIGS. 1 and 2, and the sample 20 is applied to and spread on the adhesive surface 21a. The sample 20 passes through the space 24 while adhering to the adhesive surface 21a, and is supplied to the central area 40. Once the coating plate 22 passes over the central area 40, the first step may be completed. The coating plate 22 may be moved while being inclined at a predetermined angle with respect to the adhesive surface 21a. The angle (inclination angle θ₁) formed by the coating plate 22 and the adhesive surface 21a is preferably an angle at which the coating plate 22 is inclined toward the other end 21B. For example, the inclination angle θ₁ of FIG. 2 may be 40 to 80 degrees, or may be 50 to 70 degrees. The moving speed of the coating plate 22 may be 0.1 to 10 cm/sec, or may be 0.5 to 5 cm/sec.

In the second step, for example, a ratio of the coating area of the sample 20 in the central area 40 to the total area of the central area 40 is determined. The quality can be evaluated based on the coating area proportion. The coating area proportion may be, for example, 80% or more, or may be 90% or more. The coating area of the sample 20 in the central area 40 may be determined by performing image analysis of the central area 40. In addition, the quality may be evaluated relatively by visually comparing the coating area without calculating a specific proportion.

According to the quality evaluation method of the present embodiment, it is possible to simply evaluate the quality of a hexagonal boron nitride powder or a powdery composition such as a cosmetic preparation containing the same. For example, the quality such as spreadability and fineness which are common evaluation items for cosmetic preparations and their raw materials but are difficult to standardize evaluation can be evaluated simply with high accuracy.

The position and size of the central area 40 and the space 24 and the amount of the sample 20 may be set depending on the amount of the sample 20 available and the quality of the sample. In addition, the entire sample 20 may be placed between virtual extension lines VL1 and VL2 on the one end 21A side of the adhesive surface 21a (refer to FIG. 1). Here, the virtual extension lines VL1 and VL2 are straight lines extending a pair of sides, which partition the central area 40 and are along the direction in which a hexagonal boron nitride powder is applied and spread, toward the sample 20 side. The length M of one side of the central area 40 may be, for example, 1 to 50 mm or 5 to 20 mm.

The length L of the adhesive surface 21a (tape 21) may be 5 to 200 mm, or may be 10 to 100 mm. The length M of one side of the adhesive surface 21a (tape 21) may be 5 to 100 mm, or may be 10 to 50 mm. The width of the coating plate 22 may be greater than the length M.

Ball numbers for the adhesive surface 21a may be 6 to 8 in an inclined ball tack test (inclination angle θ₁ of inclined plate: 30 degrees) specified in JIS Z 0237:2009. By performing quality evaluation using such an adhesive surface 21a, sensory evaluation of spreadability and fineness when a hexagonal boron nitride powder and a cosmetic preparation which is a raw material thereof is applied to human skin and the evaluation method of the present embodiment are more consistent, and the quality such as spreadability and fineness of them can be evaluated with high accuracy. From such a viewpoint, the ball number may be 7.

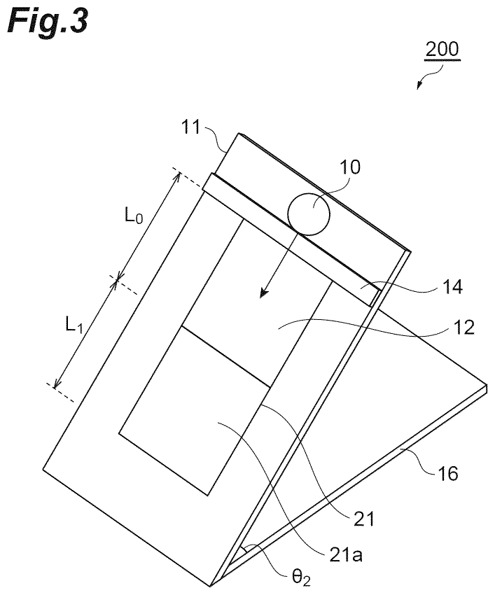

FIG. 3 is a view showing a measurement device for performing an inclined ball tack test specified in JIS Z 0237:2009. A measurement device 200 of FIG. 3 has: an inclined plate 11 that is inclined at an inclination angle θ₂ (θ₂=30 degrees) with respect to a horizontal plane; a support plate 16 that supports the inclined plate 11; and a fixing unit 14 that fixes a ball 10 to an upper end portion of the inclined plate 11. A runway 12 and the tape 21 having the adhesive surface 21a are provided on the inclined plate 11 adjacent to each other in this order from the fixing unit 14 side.

Balls 10 with various sizes are prepared, the fixing unit 14 is removed to roll the balls 10 toward the adhesive surface 21a to perform measurement. Then, among the balls 10 that stop on the adhesive surface 21a for 5 seconds or longer, a ball number having the largest size is determined. Such measurement is performed three times by replacing the tape 21, and an average value of the ball numbers corresponding to the maximum size is determined. This average value is defined as a ball number in the present disclosure. A length L₀ of the runway 12 and a length L₁ of the adhesive surface 21a are both 100 mm.

In a hexagonal boron nitride powder according to one embodiment, a coating area proportion of the adhesive surface 21a for which a ball number is 7 in an inclined ball tack test is 80% or more when the hexagonal boron nitride powder is applied to and spread on the adhesive surface 21a from the one end 21A to the other end 21B of the adhesive surface 21a using the coating plate 22 at a rate of 1 cm/sec. The coating area proportion may be 90% or more. The coating area proportion is a ratio of a coating area to a total area of the adhesive surface 21a in a central area 40 when 0.1 g of the sample 20 (hexagonal boron nitride powder) is applied to and spread on the adhesive surface 21a. 0.1 g of the entire sample 20 is placed between the virtual extension lines VL1 and VL2 of the pair of sides which partition the central area 40 and are along the direction in which the sample 20 is applied and spread.

The inclined ball tack test is as shown in FIG. 3 and its explanation, and is specified in JIS Z 0237:2009. The central area 40 in the present embodiment is a 10 mm square area. In addition, the central area 40 of FIG. 1 is 10 mm away from the one end 21A of the adhesive surface 21a. That is, a length G of the space 24 in FIG. 1 is 10 mm, and the length M of one side of the central area 40 is 10 mm.

Since the above-described hexagonal boron nitride powder has a large coating area proportion of the adhesive surface 21a, it has excellent spreadability and fineness. Further, the adhesive surface 21a for which a ball number is 7 in the above-described inclined ball tack test is similar to human skin in terms of spreadability and conformability of the hexagonal boron nitride powder. For this reason, the hexagonal boron nitride powder having a large coating area proportion on the adhesive surface 21a has excellent spreadability and fineness when particularly applied to human skin. Such a hexagonal boron nitride powder is suitable for use as a raw material for a cosmetic preparation.

The present disclosure can also provide a method of using hexagonal boron nitride as a raw material for a cosmetic preparation. A cosmetic preparation having excellent spreadability can cover a wider area of human skin when applied to and spread on human skin.

In the hexagonal boron nitride powder with the above-described coating area proportion of 80% or more, the specific surface area (N), the oxygen content, the ratio of the oxygen content to the specific surface area (N), the specific surface area (H), and the specific surface area (H) to the specific surface area (N) may be within the above-described numerical ranges. This makes it possible to sufficiently increase not only spreadability and fineness but also slipperiness.

A cosmetic preparation according to one embodiment has any of the hexagonal boron nitride powders described above. In such hexagonal boron nitride powders, adsorption of moisture onto the particle surface is suppressed, and static electricity generated on the surface can be suppressed. For this reason, it is thought that the hexagonal boron nitride powders are less likely to be agglomerated. Accordingly, the cosmetic preparation containing such hexagonal boron nitride powder have excellent spreadability.

Examples of the cosmetic preparation include foundation (powder foundation, liquid foundation, and cream foundation), face powder, point makeup, eye shadow, eyeliner, nail polish, lipstick, cheek rouge, and mascara. Among these, the hexagonal boron nitride powder is particularly well suited for foundation and eye shadow. The content of the hexagonal boron nitride powder in the cosmetic preparation is, for example, 0.1 to 70 mass %. The cosmetic preparation can be produced through well-known methods. A method for producing the cosmetic preparation includes, for example, a step of formulating and mixing the hexagonal boron nitride powder with other raw materials.

A method for producing a hexagonal boron nitride powder according to one embodiment includes: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing at least one selected from the group consisting of low crystalline hexagonal boron nitride and amorphous hexagonal boron nitride; a firing step of firing a mixed powder containing the calcined product and an aid at 1600° C. or higher and lower than 1900° C. in an atmosphere of an inert gas and/or ammonia gas to obtain a fired product; a purification step of pulverizing, washing, and drying the fired product to obtain a dry powder; and an annealing step of annealing the dry powder at a temperature of 1900° C. or higher in an atmosphere of an inert gas, ammonia gas, or a mixed gas thereof.

Examples of boron-containing compounds include boric acid, boron oxide, and borax. Examples of nitrogen-containing compounds include cyandiamide, melamine, and urea. The molar ratio between boron atoms and nitrogen atoms in a raw material powder containing boron-containing compound powder and a nitrogen-containing compound powder may be boron atoms:nitrogen atoms=2:8 to 8:2, or 3:7 to 7:3. The raw material powder may contain components other than the above-described compounds. The raw material powder may contain carbonates such as lithium carbonate and sodium carbonate as a calcination aid. In addition, the raw material powder may contain a reducing substance such as carbon.

The raw material powder containing the above-described components is calcined with, for example, an electric furnace in an inert atmosphere such as nitrogen gas, helium gas, or argon gas, an ammonia atmosphere, or a mixed gas atmosphere in which these are mixed. The calcination temperature may be 600° C. to 1300° ° C., 800° C. to 1200° ° C., or 900° ° C. to 1100° C. The calcination time may be, for example, 0.5 to 5 hours or 1 to 4 hours.

The calcined product obtained through calcining contains at least one selected from the group consisting of low crystalline hexagonal boron nitride and amorphous hexagonal boron nitride. In the calcination step, the reaction of boron nitride is allowed to progress at a lower temperature than in the firing step to be described below. Grain growth can be suppressed by lowering the calcination temperature, and the particle diameter of a boron nitride powder finally obtained can be reduced. In addition, the specific surface area (N) of the hexagonal boron nitride powder can be increased.

Next, the obtained calcined product is formulated and mixed with an aid to obtain a mixed powder. Examples of aids include borates such as sodium borate and carbonates such as sodium carbonate, calcium carbonate, and lithium carbonate. The formulation amount of the aid may be 2 to 20 parts by mass or 2 to 8 parts by mass based on 100 parts by mass of the calcined product containing hexagonal boron nitride. Such a mixed powder is fired in, for example, an electric furnace in an inert atmosphere such as nitrogen gas, helium gas, or argon gas, an ammonia atmosphere, or a mixed gas atmosphere in which these are mixed.

In the firing step, production and crystallization of boron nitride progress in the presence of the aid. As a result, the crystallinity of the boron nitride contained in the calcined product can be enhanced. The firing temperature is 1600° C. or higher and lower than 1900° C. This firing temperature may be 1650° C. to 1850° C. or 1650° C. to 1750° C. The firing time may be, for example, 0.5 to 5 hours or 1 to 4 hours.

By lowering the firing temperature, the specific surface area (N) and the specific surface area (H) can be increased. However, if the firing temperature becomes too low, there is a trend for production and crystallization of hexagonal boron nitride to be less likely to proceed sufficiently. If hexagonal boron nitride is insufficiently crystallized, there is a trend for slipperiness to decrease in a case where the hexagonal boron nitride is used in a cosmetic preparation. The same trend is observed even when the firing time is too short. On the other hand, by increasing the firing temperature, the specific surface area (N) and the specific surface area (H) are decreased. If the firing temperature becomes too high, the crystal growth of hexagonal boron nitride proceeds too much, which tends to make fine pulverization difficult. The same trend is observed even when the firing time is too long.

The fired product obtained in the firing step may be pulverized with a usual pulverization device. The pulverized powder may contain impurities in addition to hexagonal boron nitride. Examples of impurities include a residual aid and a water-soluble boron compound. In the purification step, such impurities are reduced through washing. After washing, solid-liquid separation and drying are performed to obtain a dry powder. Examples of washing liquids used for washing include water, an aqueous solution containing an acidic substance, an organic solvent, and a mixed liquid of an organic solvent and water. From the viewpoint of avoiding secondary contamination of impurities, water having an electric conductivity of 1 mS/m or less may be used.

Examples of acidic substances include inorganic acids such as hydrochloric acid and nitric acid. Examples of organic solvents include water-soluble organic solvents such as methanol, ethanol, propanol, isopropyl alcohol, and acetone. The washing method is not particularly limited. For example, the pulverized powder may be immersed in a washing liquid and stirred and washed, or the pulverized powder may be washed by spraying a washing liquid.

After the completion of washing, the washing liquid may be subjected to solid-liquid separation through decantation or using a suction filter, a pressure filter, a rotary filter, a sedimentation separator, or a combination thereof. A dry powder may be obtained by drying the separated solid content in a usual dryer. Examples of dryers include a shelf-type dryer, a fluidized bed dryer, a spray dryer, a rotary-type dryer, a belt-type dryer, and a combination thereof. After drying, for example, classification with a sieve may be performed to remove coarse particles.

In the annealing step, the dry powder is heated at 1900° C. with, for example, an electric furnace in an inert atmosphere such as nitrogen gas, helium gas, or argon gas, an ammonia atmosphere, or a mixed gas atmosphere in which these are mixed. This annealing temperature may be 1950° C. or higher or 2000° C. or higher from the viewpoint of sufficiently reducing the oxygen content. By performing the annealing step, oxygen present as functional groups or the like on the surface of particles can be dispersed to reduce the oxygen content. In the annealing step, since the dry powder with a lower content of an aid than the fired product due to the purification step is annealed, the oxygen content can be reduced while suppressing grain growth.

From the viewpoint of suppressing the grain growth, the annealing temperature may be 2200° ° C. or lower or 2100° C. or lower. The annealing time may be, for example, 0.5 to 5 hours or 1 to 4 hours from the viewpoints of sufficiently reducing the oxygen content and suppressing the grain growth.

The above-described hexagonal boron nitride powder can be obtained in this manner. The description regarding the embodiments of the hexagonal boron nitride powders can be applied to the above-described production method. In one example of a hexagonal boron nitride powder obtained through the above-described production method, a coating area proportion of the adhesive surface 21a for which a ball number is 7 in an inclined ball tack test is 80% or more when the hexagonal boron nitride powder is applied to and spread on the adhesive surface 21a from the one end 21A to the other end 21B of the adhesive surface 21a using the coating plate 22 at a rate of 1 cm/sec.

The method for producing a hexagonal boron nitride powder is not limited to the above-described embodiment. For example, the annealing step may be repeated plural times. In addition, after the annealing step, a crushing step of crushing a hexagonal boron nitride powder using a homogenizer applying ultrasound vibration may be performed.

Some embodiments of the present disclosure have been described above, but the present disclosure is not limited to any of the above-described embodiments.

### EXAMPLES

The contents of the present disclosure will be described in more detail with reference to examples and comparative examples, but the present disclosure is not limited to the following examples.

### Example 1

### [Preparation of Hexagonal Boron Nitride Powder]

### <Calcination Step>

100.0 g of a boric acid powder (purity of 99.8 mass % or more, manufactured by Kanto Chemical Co., Inc.) and 90.0 g of a melamine powder (purity of 99.0 mass % or more, manufactured by Wako Pure Chemical Industries, Ltd.) were mixed with each other with an alumina mortar for 10 minutes to obtain a mixed raw material. The mixed raw material after drying was placed in a container made of hexagonal boron nitride and placed in an electric furnace. The temperature was raised from room temperature to 1000° C. at a rate of 10° C./min while circulating nitrogen gas in the electric furnace. After holding the temperature at 1000° C. for 2 hours, the heating was stopped and the mixture was allowed to cool naturally. The electric furnace was open at a point in time when the temperature became 100° C. or lower. In this manner, a calcined product containing low crystalline hexagonal boron nitride was obtained.

### <Firing Step>

3.0 g of sodium carbonate (purity of 99.5 mass % or more) as an aid was added to 100.0 g of the calcined product and mixed with each other with an alumina mortar for 10 minutes. The mixture was placed in the above-described electric furnace. The temperature was raised from room temperature to 1700° C. at a rate of 10° C./min while circulating nitrogen gas in the electric furnace. After holding the temperature at a firing temperature of 1700ºC for 4 hours, the heating was stopped and the mixture was allowed to cool naturally. The electric furnace was open at a point in time when the temperature became 100° C. or lower. The obtained fired product was collected and pulverized with alumina mortar for 3 minutes to obtain a coarse powder of hexagonal boron nitride.

### <Purification Step>

In order to remove impurities contained in a coarse powder of hexagonal boron nitride, 30 g of the coarse powder was put into 500 g (concentration of nitric acid: 5 mass %) of dilute nitric acid and stirred at room temperature for 60 minutes. After stirring, solid-liquid separation was performed through suction filtration, and washing was performed by replacing the liquid with water (electric conductivity: 1 mS/m) until the filtrate became neutral. After washing, the solid was dried with a dryer at 120° C. for 3 hours to obtain a dry powder.

### <Annealing Step>

The dry powder was placed in the above-described electric furnace. The temperature was raised from room temperature to 2000° ° C. at a rate of 10° C./min while circulating nitrogen gas in the electric furnace. After holding the temperature at 2000° C. for 4 hours, the heating was stopped and the mixture was allowed to cool naturally. The electric furnace was open at a point in time when the temperature became 100° C. or lower. The obtained fired product was collected and pulverized in an alumina mortar for 3 minutes, and a coarse powder was removed from the obtained dry powder using an ultrasonic vibrating sieve (manufactured by Kowa Kogyosho Co., Ltd., trade name: KFS-1000, mesh size of 250 μm) to obtain a hexagonal boron nitride powder of Example 1.

### [Evaluation 1 of Hexagonal Boron Nitride Powder]

### <Measurement of Specific Surface Area (N)>

The specific surface area of the hexagonal boron nitride powder produced in Example 1 was measured through a BET 1-point method using a specific surface area measurement device (manufactured by Yuasa Ionics Co., Ltd., device name: MONOSORB). Nitrogen gas was used as an adsorption gas, and helium gas was used as a carrier gas. Measurement was performed after drying and degassing 1 g of a sample under the conditions of 300° C. for 15 minutes. The measurement results are shown in Table 2 as “Specific surface area (N).”

### <Measurement of Specific Surface Area (H)>

The hexagonal boron nitride powder produced in Example 1 was vacuum-degassed at 300° C. for 12 hours. H₂O gas was used as an adsorption gas, and a commercially available adsorption amount measurement device (manufactured by MicrotracBEL Corp., device name: BELSORP-max II) was used to measure the specific surface area (H) of the hexagonal boron nitride powder after vacuum-degassing through a BET method. The measurement results are shown in Table 2 as “Specific surface area (H).” The ratio of the specific surface area (H) to the specific surface area (N) is also concurrently shown in Table 2.

### <Measurement of Oxygen Content>

The oxygen content was measured with a simultaneous oxygen/nitrogen analyzer (manufactured by Horiba, Ltd., device name: EMGA-920). Specifically, the oxygen content and the nitrogen content were measured while heating the hexagonal boron nitride powder from room temperature to 3000° ° C. at a heating rate of 4.6° C./sec in a helium atmosphere. The oxygen content detected while no nitrogen was detected was defined as an oxygen content. The measurement results are as shown in Table 2. The ratio of the oxygen content to the specific surface area (N) is also concurrently shown in Table 2.

### [Evaluation 2 of Hexagonal Boron Nitride Powder]

### <Ball Number for Adhesive Surface>

Commercially available carbon tape (double-sided adhesive tape for SEM) was prepared as a test piece. The tackiness of the adhesive surface on one side of this carbon tape was determined through an inclined ball tack test specified in JIS Z 0237:2009. Specifically, the measurement device 200 as shown in FIG. 3 was prepared. The inclination angle θ₁ of the inclined plate 11 was set to 30 degrees, and polyethylene terephthalate film specified in JIS 2318 was pasted on the runway. The length L₀ of the runway was 100 mm, and the length L₁ of the carbon tape 21 (adhesive surface 21a) was 100 mm.

Balls having a total of 31 sizes and materials corresponding to ball numbers 2 to 32 specified in JIS Z 0237:2009 were rolled in ascending order from a start position. In a case where a ball stopped on the adhesive surface 21a for 5 seconds or longer, this was evaluated as “A,” and in a case where a ball did not stop on the adhesive surface 21a or stopped for less than 5 seconds, this was evaluated as “B.” The test was performed using three test pieces (n=3). The results are as shown in Table 1.

TABLE 1

| | | | | | | | | | Average |
|---|---|---|---|---|---|---|---|---|---|
| Ball number | 2 | 3 | 4 | 5 | 6 | 7 | 8 to 32 | Result | value |
| n = 1 | A | A | A | A | A | A | B | 7 | 7 |
| n = 2 | A | A | A | A | A | A | B | 7 | |
| n = 3 | A | A | A | A | A | A | B | 7 | |

As shown in Table 1, the ball number (average value) for the adhesive surface 21a was 7. Using the carbon tape having such an adhesive surface, the following coating area proportion evaluation was performed.

### <Evaluation of Coating Area Proportion>

As shown in FIGS. 1 and 2, the carbon tape 21 was cut into a predetermined size (width W×length L=12 mm×50 mm), and an adhesive surface on a side opposite to the adhesive surface 21a (one side) for which a ball number is 7 was pasted on the upper surface 30a of the pedestal 30. As shown in FIGS. 1 and 2, 0.1 g of the hexagonal boron nitride powder of Example 1 was placed as a sample 20 on one end 21A side of the carbon tape 21 in the longitudinal direction. At this time, the sample 20 was placed between the virtual extension lines VL1 and VL2. The coating plate 22 was moved along in the longitudinal direction of the carbon tape 21 (arrow direction in FIGS. 1 and 2) at a rate of 1 cm/sec while maintaining an angle (inclination angle θ₂) formed by the coating plate 22 and the upper surface 30a at 60 degrees, and the hexagonal boron nitride powder was applied to and spread on the adhesive surface 21a.

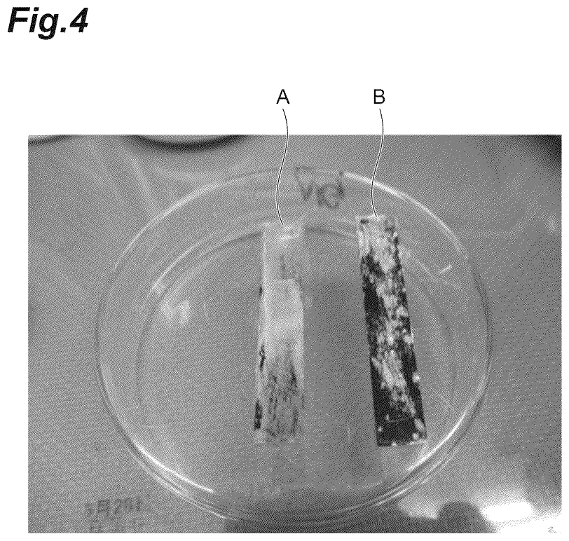

FIG. 4 is a photograph showing a state after hexagonal boron nitride powders are applied to and spread on adhesive surfaces of carbon tapes. A tape A of FIG. 4 is a photograph showing a state after the hexagonal boron nitride powder of Example 1 was applied to and spread on an adhesive surface. The coating area proportion in the central area 40 of the adhesive surface 21a shown in FIG. 1 was determined. This central area 40 is a square area of 10 mm square 10 mm away from the one end 21A and 1 mm away from the pair of side portions 21C. Image analysis was performed using commercially available image analysis software (WinROOF) to determine the ratio of the coating area of the hexagonal boron nitride powder to the total area of the central area 40. The coating area proportion is as shown in Table 3.

### [Evaluation 3 of Hexagonal Boron Nitride Powder]

### <Evaluation of Spreadability and Fineness>

0.2 g of the hexagonal boron nitride powder of Example 1 was placed on one end of artificial skin (manufactured by Idemitsu Techno Fine Co., Ltd., trade name: SUPPLALE PBZ13001 BK, length×width=10 mm×50 mm). The hexagonal boron nitride powder was spread along the surface of the artificial skin in the vertical direction using a spatula so that the hexagonal boron nitride powder was applied to the surface. Image analysis was performed using commercially available image analysis software (WinROOF) to determine the ratio of the coating area of the hexagonal boron nitride powder to the total area of the artificial skin. The larger this area proportion, the better the spreadability. The evaluation criteria for spreadability were as shown below depending on the coating area proportion.

The cross section of the artificial skin in the depth direction after the hexagonal boron nitride powder of Example 1 was applied and stretched was observed for fineness with a microscope (manufactured by Keyence Corporation, Digital Microscope VHX-7000). Then, the percentage of wrinkles (based the number of wrinkles) in the artificial skin into which the hexagonal boron nitride powder (BN) had entered was calculated. The evaluation criteria for fineness are as follows. The evaluation results of spreadability and fineness are as shown in Tables 2 and 3. The evaluation results of spreadability are shown in both Tables 2 and 3.

### [Evaluation Criteria for Spreadability]

Very good: Coating area proportion is 95% or more.
Good: Coating area proportion is 80% or more and less than 95%.
Somewhat good: Coating area proportion is 70% or more and less than 80%.
Fair: Coating area proportion is 60% or more and less than 70%.
Poor: Coating area proportion is 40% or more and less than 60%.
Very poor: Coating area proportion is less than 40%.

### [Evaluation Criteria for Fineness]

Very good: The percentage of wrinkles into which BN had entered was 85%.
Good: The percentage of wrinkles into which BN had entered was 70% or more and less than 85%.
Somewhat good: The percentage of wrinkles into which BN had entered was 70% or more and less than 80%.
Fair: The percentage of wrinkles into which BN had entered was 55% or more and less than 70%.
Not good: The percentage of wrinkles into which BN had entered was less than 55%.

### Example 2

A hexagonal boron nitride powder was prepared in the same manner as in Example 1 except that the firing temperature in the firing step was set to 1600° C. Then, various measurements and evaluations of the hexagonal boron nitride powder were performed in the same manner as in Example 1. The results were as shown in Tables 2 and 3.

### Example 3

A hexagonal boron nitride powder was prepared in the same manner as in Example 1 except that the holding time at 2000° C. in the annealing step was set to 2 hours. Then, various measurements and evaluations of the hexagonal boron nitride powder were performed in the same manner as in Example 1. The results were as shown in Tables 2 and 3.

### Example 4

A hexagonal boron nitride powder was produced in the same manner as in Example 1 except that 3.0 g of sodium carbonate (purity of 99.5 mass % or higher) was added as an aid to a mixed raw material after drying and a firing step was performed without performing a calcination step. Then, various measurements and evaluations of the hexagonal boron nitride powder were performed in the same manner as in Example 1. The results were as shown in Tables 2 and 3.

### Comparative Example 1

A powder obtained by removing coarse grains in a purification step without performing an annealing step was used as a hexagonal boron nitride powder of Comparative Example 1. Various measurements and evaluations of the hexagonal boron nitride powder were performed in the same manner as in Example 1. The results were as shown in Tables 2 and 3. A tape B of FIG. 4 is a photograph showing a state after the hexagonal boron nitride powder of Comparative Example 1 was applied to and spread on an adhesive surface 21a.

TABLE 2

| | | | Specific | | | |
|---|---|---|---|---|---|---|
| | Specific | Specific | surface | | Oxygen | |
| | surface | surface | area (H)/ | | content/specific | |
| | area | area | specific | Oxygen | surface area | |
| | (N) | (H) | surface | content | (N) | |
| | [m²/g] | [m²/g] | area (N) | [mass %] | [g/100 m²] | Spreadability |
| Example 1 | 1.91 | 0.25 | 0.13 | 0.01 | 0.005 | Very good |
| Example 2 | 5.29 | 0.51 | 0.10 | 0.10 | 0.019 | Good |
| Example 3 | 2.40 | 0.38 | 0.16 | 0.07 | 0.029 | Good |
| Example 4 | 3.75 | 0.85 | 0.23 | 0.30 | 0.080 | Somewhat good |
| Comparative | 2.20 | 0.46 | 0.21 | 0.65 | 0.295 | Very poor |
| Example 1 | | | | | | |

As shown in Table 2, it was confirmed that Examples 1 to 3 had a lower oxygen content per specific surface area (N) and a smaller ratio of the specific surface area (H) to the specific surface area (N) than Comparative Example 1. It was confirmed from the evaluation results of Examples 1 and 3 that the ratio of the specific surface area (H) to the specific surface area (N) was increased by shortening the time for the annealing step. When observing the appearance, Comparative Example 1 formed agglomerated lumps, whereas Examples 1 to 4 clearly had fewer agglomerated lumps than Comparative Example 1. For this reason, it was inferred that Examples 1 to 4 would have better spreadability than Comparative Example 1. Example 1 had particularly few agglomerated lumps and had the best spreadability.

TABLE 3

| | Coating area | | |
|---|---|---|---|
| | proportion | | |
| | [%] | Spreadability | Fineness |
| Example 1 | 95 | Very good | Very good |
| Example 2 | 82 | Good | Good |
| Example 3 | 80 | Good | Somewhat good |
| Example 4 | 77 | Somewhat good | Somewhat good |
| Comparative | 75 | Very poor | Not good |
| Example 1 | | | |

As shown in Table 3, it was confirmed that the higher the coating area proportion, the easier it was to spread, the better the spreadability was, the easier it was to enter irregularities such as wrinkles, and the better the fineness. It was confirmed also from the comparison between the tape A and the tape B in FIG. 4 that Example 1 had a higher coating area proportion than Comparative Example 1.

### INDUSTRIAL APPLICABILITY

According to the present disclosure, a hexagonal boron nitride powder having excellent spreadability and fineness is provided. In addition, a cosmetic preparation with excellent spreadability and fineness using the above-described hexagonal boron nitride powder is provided.

### REFERENCE SIGNS LIST

10: Ball, 11: Inclined plate, 12: Runway, 14: Fixing unit, 16: Support plate, 20: Sample, 21: Tape (carbon tape), 21A: One end, 21B: Other end, 21C: Side portion, 21a: Adhesive surface, 22: Coating plate, 24: Space, 30: Pedestal, 30a: Upper surface, 40: Central area, 100: Quality evaluation device, 200: Measurement device.

1. A hexagonal boron nitride powder,
wherein a ratio of an oxygen content to a specific surface area (N) determined through nitrogen adsorption is 0.1 [g/100 m²] or less.

2. A hexagonal boron nitride powder,
wherein a coating area proportion of an adhesive surface for which a ball number is 7 in an inclined ball tack test, specified in JIS Z 0237:2009, with an inclined plate with an inclination angle of 30 degrees is 77% or more when the hexagonal boron nitride powder is applied to and spread on the adhesive surface from one end to the other end of the adhesive surface using a coating plate at a rate 1 cm/sec,
where the coating area proportion is a ratio of a coating area to a total area of the adhesive surface in a central area when 0.1 g of the hexagonal boron nitride powder is applied to and spread on the adhesive surface,
the central area is a 10 mm square area 10 mm away from one end of the adhesive surface, and
the entire hexagonal boron nitride powder is arranged on one end side of the adhesive surface between virtual extension lines of a pair of sides that partition the central area along the direction in which the hexagonal boron nitride powder is applied and spread.

3. The hexagonal boron nitride powder according to claim 2,
wherein a ratio of an oxygen content to a specific surface area (N) determined through nitrogen adsorption is 0.1 [g/100 m²] or less.

4. The hexagonal boron nitride powder according to claim 1,
wherein a specific surface area (H) determined through water vapor adsorption is 0.8 [m²/g] or less.

5. The hexagonal boron nitride powder according to claim 1,
wherein a ratio of the specific surface area (H) determined through water vapor adsorption to the specific surface area (N) determined through nitrogen adsorption is 0.2 or less.

6. The hexagonal boron nitride powder according to claim 1,
wherein the oxygen content is 0.15 mass % or less.

7. The hexagonal boron nitride powder according to claim 1, which is for a raw material of a cosmetic preparation.

8. A cosmetic preparation comprising:
the hexagonal boron nitride powder according to claim 1.

9. A method for producing a hexagonal boron nitride powder, the method comprising:
a firing step of firing a mixed powder containing hexagonal boron nitride and an aid at 1600° ° C. or higher and lower than 1900° C. in an atmosphere of an inert gas, ammonia gas, or a mixed gas thereof to obtain a fired product containing hexagonal boron nitride having higher crystallinity than the hexagonal boron nitride of the mixed powder;
a purification step of pulverizing, washing, and drying the fired product to obtain a dry powder; and
an annealing step of annealing the dry powder at 1900° C. or higher in an atmosphere of an inert gas, ammonia gas, or a mixed gas thereof.

10. The method for producing a hexagonal boron nitride powder according to claim 9, further comprising, before the firing step:
a calcination step of firing a raw material powder containing a boron-containing compound powder and a nitrogen-containing compound powder at 600° C. to 1300° ° C. in an atmosphere of an inert gas, ammonia gas, or a mixed gas thereof to obtain a calcined product containing hexagonal boron nitride,
wherein the mixed powder in the firing step contains the calcined product and the aid.

11. The method for producing a hexagonal boron nitride powder according to claim 9,
wherein a ratio of an oxygen content to a specific surface area (N) determined through nitrogen adsorption in the hexagonal boron nitride powder obtained in the annealing step is 0.1 [g/100 m²] or less.

12. The method for producing a hexagonal boron nitride powder according to claim 9,
wherein, in the hexagonal boron nitride powder, a coating area proportion of an adhesive surface for which a ball number is 7 in an inclined ball tack test, specified in JIS Z 0237:2009, with an inclined plate with an inclination angle of 30 degrees is 77% or more when the hexagonal boron nitride powder is applied to and spread on the adhesive surface from one end to the other end of the adhesive surface using a coating plate at a rate of 1 cm/sec,
where the coating area proportion is a ratio of a coating area to a total area of the adhesive surface in a central area when 0.1 g of the hexagonal boron nitride powder is applied to and spread on the adhesive surface,
the central area is a 10 mm square area 10 mm away from one end of the adhesive surface, and
the entire hexagonal boron nitride powder is arranged on one end side of the adhesive surface between virtual extension lines of a pair of sides that partition the central area along the direction in which the hexagonal boron nitride powder is applied and spread.

13. A method for producing a cosmetic preparation, the method comprising:
producing the cosmetic preparation using the hexagonal boron nitride powder obtained through the method for producing according to claim 9 as a raw material.

14. A quality evaluation method comprising:
a step of applying and spreading a hexagonal boron nitride powder or a powdery composition containing the same onto an adhesive surface using a coating plate; and
a step of performing quality evaluation based on a coating area of the hexagonal boron nitride powder or the powdery composition containing the same on the adhesive surface.

15. The quality evaluation method according to claim 14,
wherein the adhesive surface is composed of one side of carbon tape.

16. The quality evaluation method according to claim 14,
wherein ball numbers for the adhesive surface are 6 to 8 in an inclined ball tack test, specified in JIS Z 0237:2009, with an inclined plate with an inclination angle of 30 degrees.

17. The hexagonal boron nitride powder according to claim 2,
wherein a specific surface area (H) determined through water vapor adsorption is 0.8 [m²/g] or less.

18. The hexagonal boron nitride powder according to claim 2,
wherein a ratio of the specific surface area (H) determined through water vapor adsorption to the specific surface area (N) determined through nitrogen adsorption is 0.2 or less.

19. The hexagonal boron nitride powder according to claim 2,
wherein the oxygen content is 0.15 mass % or less.

20. A cosmetic preparation comprising:
the hexagonal boron nitride powder according to claim 2.
